(19) United States
(12) Patent Application Publication
(10) Pub. No.: US 2022/0370986 A1
(43) Pub. Date: Nov. 24, 2022

(54) METHOD FOR THE HETEROGENEOUS CATALYSIS USING A FERROMAGNETIC MATERIAL HEATED BY MAGNETIC INDUCTION AND CATALYST SUPPORT USED FOR SAID METHOD

(71) Applicants: CENTRE NATIONAL DE LA RECHERCHE SCIENTIFIQUE, PARIS (FR); INSTITUT NATIONAL DES SCIENCES APPLIQUEES DE TOULOUSE, TOULOUSE (FR)

(72) Inventors: Julien MARBAIX, BORDEAUX (FR); Sumeet KALE, PUNE (IN); Pauline KERROUX, SAINT QUAY PERROS (FR); Juan Manuel ASENSION REVERT, TOULOUSE (FR); Stéphane FAURE, LAUNAGUET (FR); Nicolas MILLE, TOULOUSE (FR); Julian CARREY, TOULOUSE (FR); Aikaterini SOULANTIKA, CLERMONT LE FORT (FR); Bruno CHAUDRET, VIGOULET AUZIL (FR)

(21) Appl. No.: 17/761,175

(22) PCT Filed: Sep. 18, 2020
(86) PCT No.: PCT/FR2020/051625

§ 371 Date: Mar. 16, 2022

(30) Foreign Application Priority Data
Sep. 19, 2019 (FR) 19 10345

(51) Int. Cl. B01J 23/755; B01J 23/745; B01J 23/10; B01J 21/12; B01J 35/00; B01J 35/02; B01J 35/06; C07C 1/12

(52) U.S. Cl. CPC B01J 23/755; B01J 23/745; B01J 23/10; B01J 21/12; B01J 35/0033; B01J 35/026; B01J 35/06; B01J 35/023; C07C 1/12; C07C 2523/755; C07C 2523/10

(57) ABSTRACT

The invention relates to a method for the heterogeneous catalysis of a reaction for the hydrogenation of a carbon oxide in the gaseous state, such as a methanation reaction, using, in a reactor (1), carbon dioxide and gaseous dihydrogen and at least one solid catalytic compound capable of catalyzing said reaction in a given temperature range T, comprising contacting said gaseous reactant and said catalytic compound in the presence of a heating agent, and heating the heating agent to a temperature within said temperature range T. The method is characterized in that the heating agent comprises a ferromagnetic material in the form of micrometric powder and/or wires, said ferromagnetic material being heated by magnetic induction by means of a field inductor, such as a coil (2) external to the reactor (1). According to one embodiment, the catalyst support for implementing said method comprises a ferromagnetic material in the form of wires of micrometric diameters, on the surface of which metal catalyst particles are deposited.

### FIELD OF THE INVENTION

The present invention relates to the field of heterogeneous catalysis, notably a gas-solid heterogeneous catalysis process comprising the contacting of at least one gaseous reactant with a catalytic solid compound positioned on a support. The present invention also relates to the support for said catalyst.

Very many processes require heterogeneous catalysis. These catalysis processes require a step of heating, sometimes at high temperature, for the implementation of the reaction, and are therefore expensive and highly energy-consuming. Research has therefore focused on more economical solutions and notably on reactions that are less energy intensive.

### PRIOR ART

Among these solutions, international application WO 2014/162099 has proposed a heterogeneous catalysis process in which the heating is carried out by magnetic induction in order to reach the temperature necessary for the reaction. More particularly in this process, the reactant is contacted with a catalytic composition which comprises a ferromagnetic nanoparticulate component, the surface of which consists at least partially of a compound that is a catalyst for said reaction, said nanoparticulate component being heated by magnetic induction in order to reach the desired temperature range. This heating may be carried out by means of a field inductor external to the reactor. In this system, the nanoparticles are heated by their own magnetic moment, enabling the startup of the catalytic reaction. The heating is therefore initiated within the very heart of the reactor, rapidly with minimal energy input. This results in substantial savings.

However, the cost of these reactions still remains high, due in particular the cost of the catalytic particles in nanometric form and more particularly the magnetic nanoparticles. Moreover, these nanomaterials must, in general, be handled with caution.

Another problem linked to the use of nanoparticles is the modification of their heating properties due, on the one hand, to their tendency toward sintering during high-temperature reactions, and, on the other hand, to aging resulting from a change in the chemical order in said nanoparticles (modification of the structure and of the local chemical composition).

### OBJECTIVES OF THE INVENTION

A first objective of the invention is therefore to overcome the aforementioned drawbacks by further reducing the cost of these heterogeneous catalysis reactions, while maintaining the reaction performance thereof.

Another objective of the invention is to propose a process that makes it possible to reduce the proportion of the components in the form of nanometric particles in the reactor.

Another objective of the invention is to propose a heterogeneous catalysis process that exhibits a maintenance of the heating properties and of the catalytic properties over very long periods of time, while being suitable for intermittent operation.

Another objective of the invention is to propose a process for catalysis of a gas-solid chemical reaction, more particularly of a hydrogenation reaction of a carbon oxide in the gaseous state, such as a methanation reaction.

### DESCRIPTION OF THE INVENTION

In the search for new savings, the inventors discovered, surprisingly, that the heating agent may not necessarily be in nanometric form, but may be present in the reactor in the form of micrometric powder or of wires.

For this purpose, the present invention proposes a process for heterogeneous catalysis of a hydrogenation reaction of a carbon oxide in the gaseous state, such as a methanation reaction using, in a reactor, carbon dioxide and gaseous dihydrogen and at least one catalytic solid compound capable of catalyzing said reaction in a given temperature range T, comprising the contacting of said gaseous reactant and of said catalytic compound in the presence of a heating agent, and the heating of the heating agent to a temperature within said temperature range T, the process is characterized in that the heating agent comprises a ferromagnetic material in the form of micrometric powder composed of micrometric ferromagnetic particles having sizes of between 1 μm and 1000 μm and/or of wires based on iron or on an iron alloy, preferably having a wire diameter of between 10 micrometers and 1 millimeter, said ferromagnetic material being heated by magnetic induction by means of a field inductor external to the reactor, the magnetic field generated by the field inductor external to the reactor having an amplitude of between 1 mT and 80 mT and a frequency of between 30 kHz and 500 kHz. The results obtained with such a heating agent which is no longer nanometric, but of much greater size, are equivalent to those obtained in the process of WO 2014/162099 with a ferromagnetic nanoparticulate component.

According to a first embodiment of the invention, when it is present in powder form, the ferromagnetic material is advantageously composed of micrometric ferromagnetic particles having sizes of between 1 μm and 100 μm, preferably between 1 μm and 50 μm, more preferably between 1 μm and 10 μm.

With such micrometric ferromagnetic particles, which admittedly sometimes have a tendency toward agglomeration, no sintering is observed and the effectiveness of the heating is thus maintained.

As regards the catalytic compound used in the process according to the invention, said catalytic compound comprises a catalyst for the heterogeneous catalysis reaction that is in the form of metallic particles positioned on a support.

Said metallic catalyst particles are advantageously chosen from manganese, iron, nickel, cobalt, copper, zinc, ruthenium, rhodium, palladium, iridium, platinum, tin, or an alloy comprising one or more of these metals.

Said metallic catalyst particles are positioned at the surface of an oxide forming a support for the catalyst, such as an oxide of at least one of the following elements: silicon, cerium, aluminum, titanium or zirconium, (for example Al₂O₃, SiO₂, TiO₂, ZrO₂, CeO₂) constituting a catalyst-oxide assembly that is in the form of a powder of micrometric or nanometric size which is mixed with the ferromagnetic material in the form of micrometric powder. The mixing of these powders (catalyst-oxide assembly with the microparticulate ferromagnetic material) thus creates intimate contact between the heating agent and the catalyst, making it possible to rapidly start the catalysis reaction at the surface of the catalyst.

According to a second embodiment of the invention, the support for the catalyst is said ferromagnetic material that is in the form of wires.

Advantageously, the ferromagnetic material that is in the form of wires, which are supports for the catalyst, may comprise, or predominantly consist of, steel wool, containing wires based on iron or on an iron alloy, preferably having a wire diameter of between 20 μm and 500 μm, more preferably between 50 μm and 200 μm.

Indeed, quite surprisingly, steel wool, a cheap and readily available material that can be purchased in home improvement stores, has proved to be an excellent heating agent. More particularly, very fine (superfine) steel wool, having a wire diameter of less than a millimeter, is both a good catalyst support and effective for enabling the heating of said catalyst by magnetic induction.

This material is very easy to use and has a very long service life. Furthermore, it is easily recyclable and is non-polluting.

The process according to the invention is advantageously a hydrocarbon synthesis reaction, more particularly the heterogeneous catalysis reaction is.

The heterogeneous catalysis process according to the invention, hydrogenation reaction of a carbon oxide in the gaseous state, such as a methanation reaction starting from carbon dioxide and dihydrogen, may in particular be carried out with a magnetic field generated by the field inductor external to the reactor having an amplitude of between 1 mT and 50 mT and a frequency of between 50 kHz and 400 kHz, preferably between 100 kHz and 300 kHz.

The present invention also relates to a catalyst support for the implementation of the heterogeneous catalysis process described above, characterized in that it comprises a ferromagnetic material in the form of wires of micrometric diameters, deposited at the surface of which are metallic catalyst particles.

Advantageously, the ferromagnetic material is based on iron, or on an iron alloy, preferably comprising at least 50 wt % iron, more preferably at least 80 wt % iron.

This ferromagnetic material may in particular be composed of superfine steel wool, comprising an entanglement of wires composed of at least 90 wt % iron, and of which the diameter of the wires is between 10 μm and 1 mm, preferably between 20 μm and 500 μm, more preferably between 50 μm and 200 μm.

### BRIEF DESCRIPTION OF THE DRAWINGS

The invention will be clearly understood on reading the following description of non-limiting exemplary embodiments with reference to the appended drawings in which:

FIG. 1A is a simplified partial diagram of a reactor for the implementation of the gas-solid heterogeneous catalysis process according to the invention, under an upward gas flow, showing the positioning of the catalyst+heating agent assembly in the part of the tubular reactor encircled by the external magnetic field inductor,

FIG. 1B is a simplified partial diagram of a reactor for the implementation of the gas-solid heterogeneous catalysis process according to the invention, under a downward gas flow, showing the positioning of the catalyst+heating agent assembly in the part of the tubular reactor encircled by the external magnetic field inductor,

FIG. 2 is a graph comparing the performance of various heating agents according to the invention, carried out under argon at 100 kHz (specific absorption rate, SAR, corresponding to the amount of energy absorbed per unit mass, expressed in watts per gram of material, as a function of the alternating magnetic field intensity applied, expressed in mT): iron powder having microparticles with a size of the order of 3-5 μm, fine steel wool (wire diameter of greater than 1 mm) and superfine steel wool (wire diameter of less than 1 mm, of the order of 100 μm),

FIG. 3 is a graph presenting results of a methanation process according to the invention using iron powder as heating agent and an Ni on SiRAIOx® ((silicon aluminum oxide from SESAL) catalyst,

FIG. 4 is a histogram showing the conversion rates (in %) of CO₂ and of CH₄ and also the selectivity as a function of time and temperature for a methanation reaction in downward flow in the presence of a mixture of iron powder and Ni/CeO₂,

FIG. 5 is a histogram showing the conversion rates (in %) of CO₂ and of CH₄ and also the selectivity as a function of time and temperature for a methanation reaction in downward flow in the presence of a mixture of steel wool and Ni/CeO₂,

FIG. 6 is a histogram showing the conversion rates (in %) of CO₂ and of CH₄ and also the selectivity as a function of time and temperature for a methanation reaction in downward flow in the presence of nickel on steel wool,

FIG. 7 is a graph comparing the energy efficiency (expressed in %) as a function of temperature for the three types of catalyst beds (catalyst+heating agent) tested in the examples presented in FIGS. 4, 5 and 6.

### EXAMPLES

### Example 1: Preparation of the Catalyst

Preparation of the Catalyst on Cerium Oxide Support

Nickel at 10 wt % on cerium oxide (abbreviated to Ni(10 wt %)/CeO₂) is prepared by decomposition of Ni(COD)₂ in the presence of CeO₂ in mesitylene.

According to a conventional preparation process, 1560 mg of Ni(COD)₂ are dissolved in 20 mL of mesitylene then 3 g of CeO₂ are added. The mixture obtained is heated at 150° C. under an argon atmosphere for 1 hour with vigorous stirring. This mixture, initially milky white, is black at the end of the reaction. After decantation, the translucent supernatant is removed and the particles obtained are washed three times with 10 mL of toluene. The toluene is then removed under vacuum, making it possible to obtain a thick powder of Ni10 wt %/CeO₂ (3.5 g) which is collected and stored in a glove box. Analysis by inductively coupled plasma mass spectrometry (ICP-MS) confirms the loading of 9 wt % of nickel (10% targeted) of the cerium oxide. Observation by transmission electron microscopy (TEM) and EDS analysis show the presence of small monodisperse particles of nickel (with a size of 2-4 nm).

Process for Preparing Ni on SiRAIOx®

In a Fischer-Porter bottle and under an inert atmosphere, 0.261 g of Ni(COD)₂ is dissolved in 20 mL of mesitylene and 0.500 g of SiRAIOx® is added. The mixture is heated at 150° C. for one hour with stirring. After returning to ambient temperature, the powder is left to precipitate, then the supernatant is removed and the powder is washed three times with 10 mL of THF. The powder is then dried under vacuum and stored under an inert atmosphere.

Mixture of Iron Powder+Ni/CeO₂

2 g of iron powder are mixed with 1 g of nickel catalyst deposited on cerium oxide prepared previously. Observation with a scanning electron microscope and also EDS mapping make it possible to visualize grains of iron powder having a size of the order of 3-5 μm and to confirm that the nickel is indeed present on the cerium oxide CeO₂.

### Example 2: Preparation of the Catalyst on Steel Wool Support

Superfine steel wool (Gerlon, purchased from Castorama). ICP-MS analysis of the superfine steel wool gives a composition of 94.7 wt % of iron. EDS mapping shows the presence of numerous impurities on the surface of the wool (mainly potassium, manganese, silicon). SEM observation makes it possible to determine the diameter of the wires of the superfine steel wool used, which is around 100 μm and has a rough and uneven surface.

The experimental protocol for depositing nickel metal on superfine steel wool (entanglement of wires of around 100 μm in diameter, containing 94.7 wt % of iron) is substantially the same as on CeO₂. 1560 mg of Ni(COD)₂ are dissolved in 100 mL of mesitylene in order to completely submerge the steel wool (3 g). After one hour under rapid stirring at 150° C. under argon, the mixture is placed in a glove box and the solution (of black color) is drained off. The steel wool has itself also turned black. The steel wool is then rinsed with toluene, and then dried under vacuum for 30 minutes and stored in a glove box. Observation by scanning electron microscopy (SEM) and energy-dispersive x-ray spectroscopy show the deposition of polydisperse particles of nickel (100 nm-1000 nm) on the surface of the wires of the steel wool.

ICP-MS analysis over three different zones shows different nickel loadings: 1.23%, 1.44% and 1.33% (weight percentages). These differences between these loadings are quite small, the surface of the wool appears homogeneous. Despite everything, the amount of nickel deposited is below the targeted percentage of 10 wt % of Ni.

### Example 3: Methanation Reaction: Measurements of Conversion and Calculation of the Selectivity

The methanation reaction

CO₂.+.4.H₂.→.CH₄.+.2.H₂O. [Chem. 1]

which is a combination of

.CO₂+.H₂. ↔CO+.H₂O. [Chem. 2]

and of

CO.+3.H₂.→.CH₄.+.H₂O. [Chem. 3]

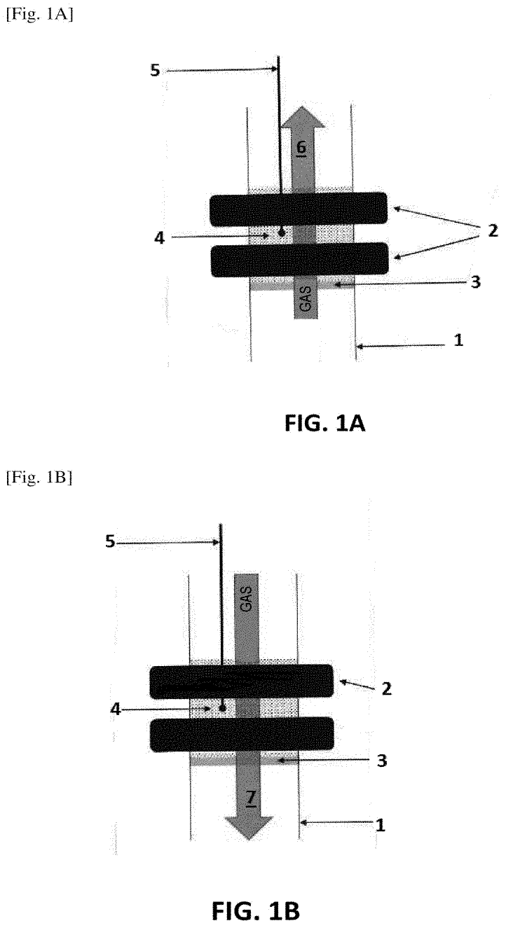

is carried out in a quartz fixed-bed tubular continuous reactor 1 (Avitec) (internal diameter: 1 cm with a height of catalyst bed 4, dependent on the heating element, of around 2 cm, resting on sintered glass 3) (cf. FIG. 1); the gaseous stream may be in upward flow 6 (FIG. 1A) or in downward flow 7 (FIG. 1B)). The coil 2 (from the company Five Celes) used is a solenoid with an internal diameter of 40 mm and a height of 40 mm that constitutes the external magnetic field inductor connected to a generator. Its resonance frequency is 300 kHz with a magnetic field varying between 10 and 60 mT. The coil 2 is water cooled.

The measurements of the conversion rates and selectivity as a function of the temperature are carried out with temperature servocontrol of the generator associated with the coil 2. For this purpose, a temperature probe 5 connected to the generator is submerged in the catalyst bed (heating agent+catalyst assembly). The generator sends a magnetic field in order to reach the fixed temperature and then only sends pulses to maintain this temperature. The reaction is carried out at atmospheric pressure and at a temperature that varies between 200° C. and 400° C. The reactor 1 is supplied with H₂ and CO₂, the flow rate of which is controlled by a flowmeter (Brooks flowmeter) and controlled by Lab View software. The proportions are the following: an overall constant flow rate of 25 mL/min comprises 20 mL/min of H₂ and 5 mL/min of CO₂. The supplying is carried out at the top of the reactor, the water formed is condensed at the bottom of the reactor (without condenser) and is recovered in a round-bottomed flask. The methane formed and the remaining gases (CO₂ and H₂) and also the CO are sent to a gas chromatography column (Perkin Elmer, Clarus 580 GC column). The conversion of the CO₂, the selectivity of the CH₄ and the yield of CO and of CH₄ are calculated according to the following equations:

$$[ Math . 2 ]$$

$$X ( {CO}_{2} ) = {CO}_{2} conversion = \frac{\begin{matrix}( FC ( CO ) \times A ( CO ) + \\ FC ( C {H}_{4} ) \times A ( C {H}_{4} )\end{matrix}}{\begin{matrix}( FC ( CO ) \times A ( CO ) + \\ FC ( C {H}_{4} ) \times A ( C {H}_{4} ) + A ( {CO}_{2} )\end{matrix}}$$

$$Y ( CO ) = CO yield = \frac{( F C ( CO ) \times A ( CO )}{\begin{matrix}( FC ( CO ) \times A ( CO ) + \\ FC ( C {H}_{4} ) \times A ( C {H}_{4} ) + A ( {CO}_{2} )\end{matrix}}$$

$$Y ( {CH}_{4} ) = {CH}_{4} yield = \frac{F C ( C {H}_{4} ) \times A ( C {H}_{4} )}{\begin{matrix}( FC ( CO ) \times A ( CO ) + \\ FC ( C {H}_{4} ) \times A ( C {H}_{4} ) + A ( {CO}_{2} )\end{matrix}}$$

$$S ( {CH}_{4} ) = {CH}_{4} selectivity = \frac{F C ( C {H}_{4} ) \times A ( C {H}_{4} )}{\begin{matrix}( FC ( CO ) \times A ( CO ) + \\ FC ( C {H}_{4} ) \times A ( C {H}_{4} )\end{matrix}}$$

$$With FC ( CO ) = 1.61 and FC ( C {H}_{4} ) = 1.71 .$$

FC is the response factor for each reactant according to reaction monitoring by gas chromatography,

A is the area of the peak measured in chromatography.

Measurements of the energy efficiency:

Energy efficiency measurements are carried out at the same time as the conversion and selectivity measurements of the methanation reaction. The electricity consumption data for the coil 2 are recovered by means of software developed in the laboratory. The energy efficiency is then calculated according to the following method

$$\begin{matrix}{n}_{therm - NRJ} = \frac{{Y}_{CH 4} . {D}_{m , CH4} . {PCS}_{CH 4}}{{D}_{m . H 2} . {PCS}_{H 2} + {E}_{bobine}} & \end{matrix} \qquad [ Math . 2 ]$$

PCS (gross calorific value) represents the amount of energy released by the combustion of 1 mg of gas.
The values given by the literature are PCS_{H2}=141.9 MJ/kg and PCS_{CH4}=55.5 MJ/kg,
Y_{CH4} being the CH₄ yield of the reaction,
Dₘᵢ being the mass flow rate of the product i,
E_{bobine} corresponds to the energy consumed by the inductor in order to operate (namely, to generate the magnetic field and cool the system).

The energy efficiency is expressed in % in FIG. 7.

### Example 4: Comparison of Various Heating Agents

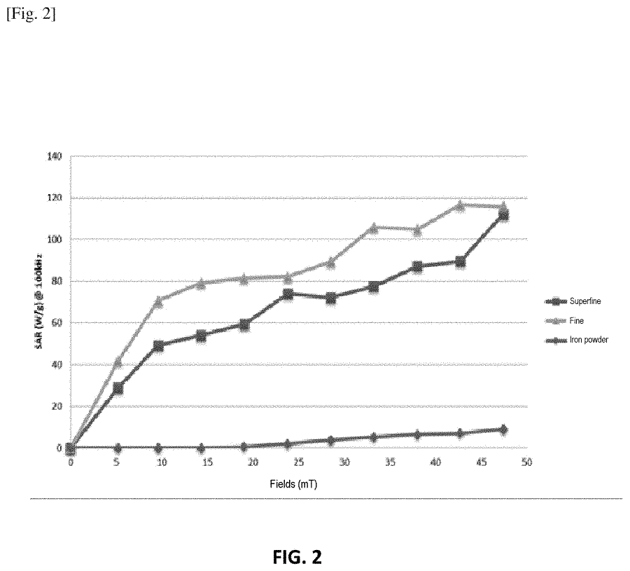

These results differ notably from those obtained in the recent publication by Kale et al., Iron carbide or iron carbide/cobalt nanoparticles for magnetically-induced CO₂ hydrogenation over Ni/SiRAIOx catalysts, Catal. Sci. Technol., 2019, 9, 2601., which reports, for the FeC nanoparticles, SAR values of between 1100 and 2100 W/g at 100 kHz. FIG. 2 shows that for a microparticulate ferromagnetic material such as iron powder or steel wool, these values are 10 to 20 times lower.

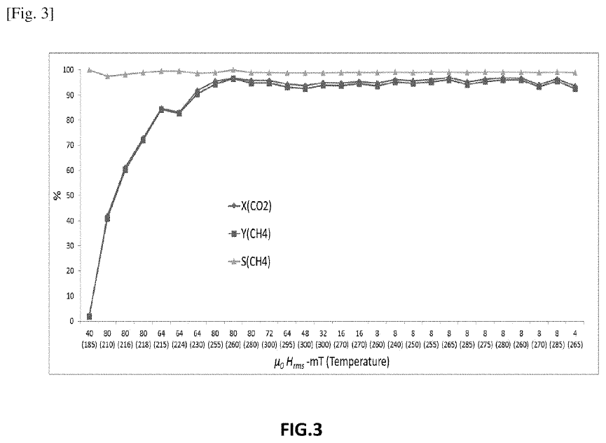

It might then be expected to have to provide the microparticulate iron powder and the steel wool with a higher field than for the nanoparticles. But the results from FIG. 3 show that this is not the case. For the iron carbide nanoparticles, it is necessary to provide a field of around 48 mT to achieve a yield close to 90%. With the iron powder, after launching the reaction, a field of only 8 mT is necessary. The distinctive feature of the iron powder and of the steel wool lies in the eddy currents that come into play and lead to a reduction of the magnetic field for heating the material.

The micrometric iron powder and the micrometric steel wool therefore constitute advantageous ferromagnetic materials for in situ heating, by magnetic induction, of the reactors carrying out gas-solid catalytic reactions such as methanation reactions starting from carbon dioxide and dihydrogen, which is presented in the following examples.

### Example 5: Mixture of Iron Powders and of Catalyst

The catalyst bed consists of nickel particles on cerium oxide: Ni: 0.09 g/CeO2: 0.91 g, mixed with 2 g of iron powder. The gas flow is downward, at a constant flow rate of 20 mL/min of H₂ and 5 mL/min of CO₂.

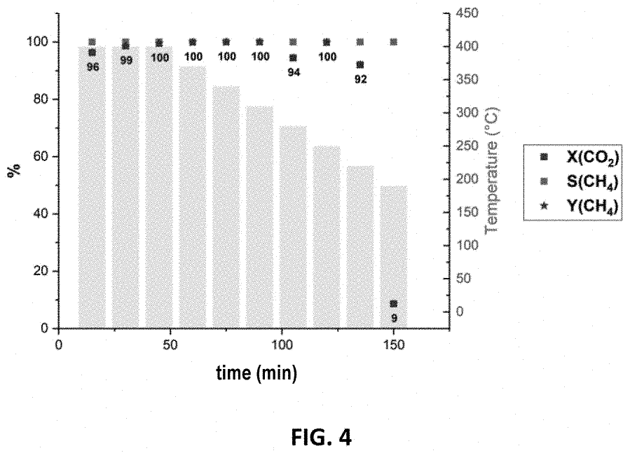

The results of the conversion rates of CO₂ and of CH₄ are presented in FIG. 4. This assembly of powders (iron powder+Ni/CeO₂) makes it possible to obtain very satisfactory yields (Y(CH₄)), reaching 100% at temperatures of 300-350° C.

### Example 6: Mixture of Steel Wool and Ni/CeO₂ Catalyst

The catalyst bed consists of nickel particles deposited on cerium oxide: Ni: 0.09 g/CeO2: 0.91 g and of 0.35 g of (superfine) steel wool. The gas flow is downward, at a constant flow rate of 20 mL/min of H₂ and 5 mL/min of CO₂.

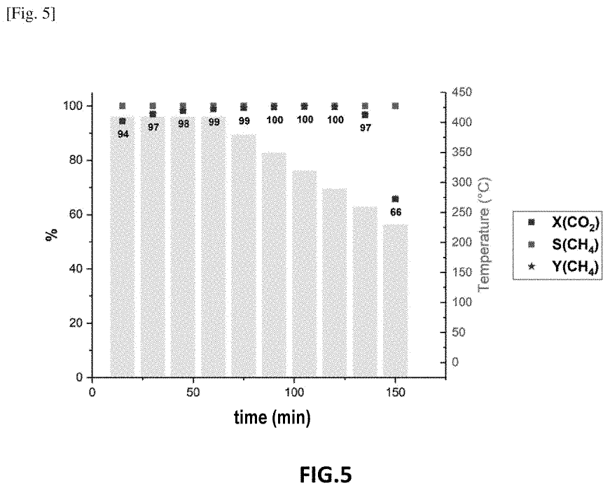

The results of the conversion rates of CO₂ and of CH₄ are presented in FIG. 5. This steel wool+Ni/CeO₂ assembly also makes it possible to obtain very satisfactory yields (Y(CH₄)), reaching 100% at temperatures of 300-350° C.

### Example 7: Ni Catalyst Deposited on Steel Wool

The catalyst bed consists of nickel particles: Ni: 0.03 g deposited on 2.27 g of (superfine) steel wool. The gas flow is downward, at a constant flow rate of 20 mL/min of H₂ and 5 mL/min of CO₂.

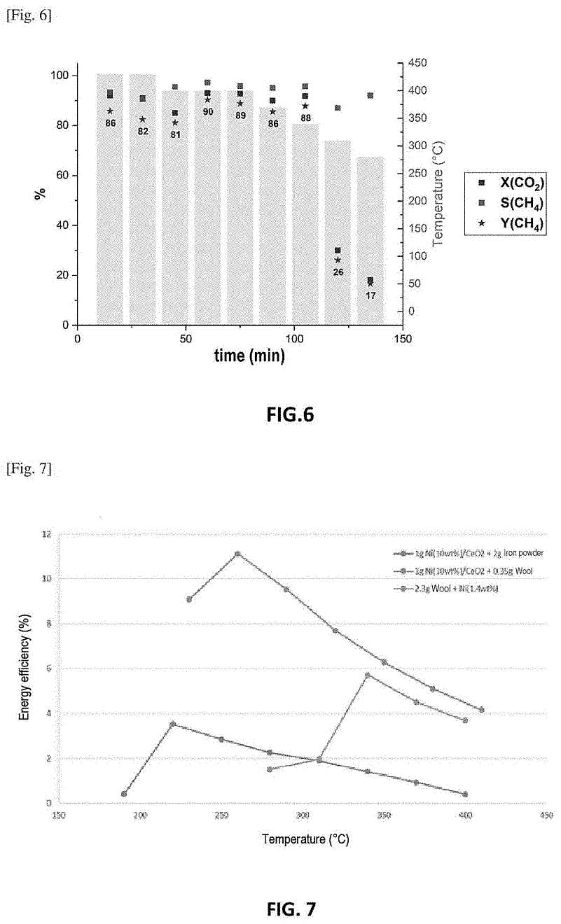

The results of the conversion rates of CO₂ and of CH₄ are presented in FIG. 6. The maximum yield (Y(CH₄)) is 90% at 400° C. This result is very encouraging, knowing that this system is simpler to implement.

### Example 8: Energy Efficiency

The energy efficiency calculations of the preceding three examples (examples 5, 6 and 7) grouped together in FIG. 7 show that it is necessary to provide less energy to the steel wool system than to the iron powder system in order to reach the same temperature. This difference between powder and wool is observed particularly with the steel wool+Ni/CeO₂ system. The energy efficiency of the steel wool+Ni is not as good since there is more wool to heat and therefore more energy to provide for a same amount of methane produced. In the example presented, it was necessary to introduce a large amount of steel wool, since very little nickel had been deposited thereon, in order to achieve an advantageous yield (90%).

1. A process for heterogeneous catalysis of a hydrogenation reaction of a carbon oxide in the gaseous state, using, in a reactor, carbon dioxide and gaseous dihydrogen and at least one catalytic solid compound capable of catalyzing said reaction in a given temperature range T, said method comprising:
contacting of said gaseous reactant and of said catalytic compound in the presence of a heating agent, and
heating of the heating agent to a temperature within said temperature range T, wherein the heating agent has a ferromagnetic material in the form of micrometric powder composed of micrometric ferromagnetic particles having sizes of between 1 μm and 1000 μm and/or of wires based on iron or on an iron alloy, said ferromagnetic material being heated by magnetic induction by means of a field inductor external to the reactor, the magnetic field generated by the field inductor external to the reactor having an amplitude of between 1 mT and 80 mT and a frequency of between 30 kHz and 500 kHz.

2. The process as claimed in claim 1, wherein the ferromagnetic material in powder form is composed of micrometric ferromagnetic particles having sizes of between 1 μm and 100 μm.

3. The process as claimed in claim 1, wherein the ferromagnetic material in powder form is composed of ferromagnetic particles, having sizes of between 1 μm and 50 μm.

4. The process as claimed in claim 1, wherein the catalytic compound comprises a catalyst for the heterogeneous catalysis reaction that is in the form of metallic particles positioned on a support.

5. The process as claimed in claim 4, wherein said metallic catalyst particles are chosen from the group consisting of manganese, iron, nickel, cobalt, copper, zinc, ruthenium, rhodium, palladium, iridium, platinum, tin, and an alloy comprising one or more of these metals.

6. The process as claimed in claim 4, wherein the metallic catalyst particles are positioned at the surface of an oxide forming a support for the catalyst, constituting a catalyst-oxide assembly that is in the form of a powder which is mixed with the ferromagnetic material in powder form.

7. The process as claimed in claim 4, wherein the support for the catalyst is said ferromagnetic material that is in the form of wires.

8. The process as claimed in claim 7, wherein the ferromagnetic material that is in the form of wires, which are the supports for the catalyst, comprises steel wool, containing wires based on iron or on an iron alloy.

9. The process as claimed in claim 1, wherein the magnetic field generated by the field inductor external to the reactor has an amplitude of between 1 mT and 50 mT.

10. The process as claimed in claim 1, wherein the magnetic field generated by the field inductor external to the reactor has a frequency of between 50 kHz and 400 kHz.

11. A catalyst support for the implementation of the process as claimed in claim 7, wherein said catalyst comprises a ferromagnetic material in the form of wires of micrometric diameters, deposited at the surface of which are metallic catalyst particles.

12. The catalyst support as claimed in claim 11, wherein the ferromagnetic material is based on iron or on an iron alloy.

13. The support as claimed in claim 11, wherein the ferromagnetic material is composed of superfine steel wool, comprising an entanglement of wires composed of at least 90 wt % iron, and of which the diameter of the wires is between 10 μm and 1 mm.

14. The process as claimed in claim 1, wherein said wires based on iron or on an iron alloy have a wire diameter of between 10 micrometers and 1 millimeter.

15. The process as claimed in claim 3, wherein the ferromagnetic material in powder form is composed of ferromagnetic particles, having sizes of between 1 μm and 10 μm.

16. The process as claimed in claim 6, wherein said oxide is an oxide selected from the group of following elements consisting of: silicon, cerium, aluminum, titanium or zirconium.

17. The process as claimed in claim 8, wherein said wires based on iron or on an iron alloy have a wire diameter of between 20 μm and 500 μm.

18. The process as claimed in claim 8, wherein said wires based on iron or on an iron alloy have a wire diameter of between 50 μm and 200 μm.

19. The process as claimed in claim 10, wherein the magnetic field generated by the field inductor external to the reactor has a frequency of between 100 kHz and 300 kHz.

20. The catalyst support as claimed in claim 12, wherein the ferromagnetic material is based on iron or on an iron alloy comprising at least 50 wt % iron.

21. The catalyst support as claimed in claim 12, wherein the ferromagnetic material is based on iron or on an iron alloy comprising at least 80 wt % iron.

22. The support as claimed in claim 13, wherein the ferromagnetic material is composed of superfine steel wool, comprising an entanglement of wires composed of at least 90 wt % iron, and of which the diameter of the wires is between 20 μm and 500 μm.

23. The support as claimed in claim 13, wherein the ferromagnetic material is composed of superfine steel wool, comprising an entanglement of wires composed of at least 90 wt % iron, and of which the diameter of the wires is between 50 μm and 200 μm.
